(19) United States
(12) Patent Application Publication
(10) Pub. No.: US 2004/0161465 A1
(43) Pub. Date: Aug. 19, 2004

(54) Absorbable nitrogenous epsilon-caprolactone copolymers and applications thereof

(76) Inventor: Shalaby W. Shalaby, Anderson, SC (US)

Correspondence Address: Nany A. Bird, 231 Walton Ave, South Orange, NJ, 07079, US

(21) Appl. No.: 10/778,695

(22) Filed: Feb. 13, 2004

Related U.S. Application Data
(63) Continuation-in-part of application No. 10/087,512, filed on Mar. 1, 2002, now Pat. No. 6,703,035.
Application No. 10/087,512 is a continuation-in-part of application No. 09/713,860, filed on Nov. 16, 2000, now Pat. No. 6,485,749.
Application No. 09/713,860 is a division of application No. 09/103,142, filed on Jun. 29, 1998, now Pat. No. 6,197,320.
Application No. 09/103,142 is a continuation-in-part of application No. 08/660,089, filed on Jun. 3, 1996, now Pat. No. 5,773,563.
Application No. 08/660,089 is a continuation of application No. 08/212,174, filed on Mar. 11, 1994, now Pat. No. 5,522,842.

(51) Int. Cl. A61K 9/14

(52) U.S. Cl. 424/486

(57) ABSTRACT

This invention deals with monocentric, triaxial, nitrogenous ε-caprolactone copolyester for coating/sizing absorbable, fibrous vascular devices wherein the said copolyesters have a molecular weight of more than 20 kDa. The invention also relates to medical devices such a absorbable micro-threads as in small diameter suture braids or twisted multifilament yarn coated with absorbable, nitrogenous ε-caprolactone copolyester wherein the copolyester chain bears one or more carboxyl groups to which a basic polyamino acid, such as polylysine or polyarginine, is linked ionically and/or covalently.

This application is a continuation-in-part of U.S. Ser. No. 10/087,512 entitled, “Absorbable ε-Caprolactone Copolymers,” filed on Mar. 1, 2002, which is a continuation-in-part of U.S. Ser. No. 09/713,860 entitled, “Absorbable ε-Caprolactone Copolymers and Medical Devices,” filed Nov. 16, 2000, now U.S. Pat. No. 6,485,749, which is a Divisional of U.S. Ser. No. 09/103,142 entitled, “Absorbable ε-Caprolactone Copolymers and Medical Devices,” filed Jun. 29, 1998, now U.S. Pat. No. 6,197,320, which is a continuation-in-part of U.S. Ser. No. 8/660,089 entitled, “Absorbable ε-Caprolactone Copolymers,” filed Jun. 3, 1996, now U.S. Pat. No. 5,773,563, which is a continuation of U.S. Ser. No. 08/212,174 entitled, “Absorbable ε-Caprolactone Copolymers as Suture Coating Displaying Autocatalyzed Hydrolysis,” filed Mar. 11, 1994, now U.S. Pat. No. 5,522,842.

### FIELD OF THE INVENTION

### BACKGROUND OF THE INVENTION

Prior applications and pertinent issued patents of the inventor have dealt with nitrogenous copolyesters based primarily on ε-caprolactone and on their use as lubricious coatings for medical devices, particularly absorbable suture braids and stents (U.S. Pat. Nos. 5,522,842; 5,773,563; 6,197,320; 6,485,749, and allowed application U.S. Ser. No. 10/087,512) as well as high strength copolyesters for biomedical constructs (U.S. Ser. No. 10/128,121, entitled “High Strength Nitrogenous Caprolactone Copolymers and Biomedical Constructs Therefrom,” filed on Apr. 23, 2002). All the prior copolyesters of the inventor, regardless of their applications and including those used as coatings for medical devices, were distinguished from prior art polyesters in that they bear nitrogenous moieties that contribute to their ability to undergo autocatalyzed hydrolysis and accelerated absorption profiles in the biological environment by virtue of having these different types of nitrogenous chemical entities incorporated and contained therein. However, the inventor's prior coating copolyesters did not exploit the combined built-in features of autocatalytic hydrolyzability, optimum surface energy, and ability to be positively charged in the biological environment of most of these copolymers. This prompted the exploration of new nitrogenous polyesters based primarily on ε-caprolactone for novel uses to improve the performance of absorbable medical devices and/or create new applications thereof. And this invention relates to the synthesis of new nitrogenous ε-caprolactone copolyester compositions for use as matrices of fibrous composites and/or active surfaces for achieving hemostasis.

### SUMMARY OF THE INVENTION

This invention deals with an absorbable, monocentric, triaxial, crystalline nitrogenous ε-caprolactone copolyester coating for a fibrous vascular device wherein the polymer molecular weight exceeds 20 kDa and the chain sequences consist of 85 to 97 percent caprolactone-derived units and 3 to 15 percent units derived from one or more cyclic monomer selected from the group represented by glycolide, morpholine-2,5-dione, and 6,6′-dimethyl morpholine-2,5-dione, covalently linked to a central nitrogen of a tertiary amine, wherein the fibrous vascular device comprises a micro-thread comprising absorbable, twisted or braided multifilament yarn having at least 10 percent by weight of said coating. This invention also relates to a medical device comprising an absorbable, crystalline nitrogenous α-caprolactone copolyester wherein the polymer molecular weight is 2 to 40 kDa and chain sequences consisting of 85 to 97 percent of caprolactone-derived units and 3 to 15 percent units derived from one or more cyclic monomer selected from the group represented by glycolide, morpholine-2,5-dione, and 6,6′-dimethyl morpholine-2,5-dione, and said copolyester has one or more carboxyl group per chain to which is ionically or covalently bound a polyamino acid having at least five hydrogen-bearing amine group(s) in sequence per chain, wherein the polyamino acid is a polylysine or polyarginine having a molecular weight of at least 1 kDa and the said device is a hemostatic vascular sealant or capable of attaining hemostasis.

Another aspect of this invention deals with a medical device comprising an absorbable, crystalline nitrogenous ε-caprolactone copolyester wherein the polymer molecular weight is 2 to 40 kDa and chain sequences consisting of 85 to 97 percent of caprolactone-derived units and 3 to 15 percent units derived from one or more cyclic monomer selected from the group represented by glycolide, morpholine-2,5-dione, and 6,6′-dimethyl morpholine-2,5-dione, and said copolyester has one or more carboxyl group per chain to which is ionically or covalently bound a polyamino acid having at least five hydrogen-bearing amine group(s) per chain, wherein the said device is a hemostatic vascular device comprising a fibrous construct comprising a micro-thread comprising an absorbable, braided or twisted multifilament yarn having at least 10 percent by weight of the nitrogenous copolyester as a coating.

Another aspect of this invention deals with a medical device comprising an absorbable, crystalline nitrogenous ε-caprolactone copolyester wherein the polymer molecular weight is 2 to 40 kDa and chain sequences consisting of 85 to 97 percent of caprolactone-derived units and 3 to 15 percent units derived from one or more cyclic monomer selected from the group represented by glycolide, morpholine-2,5-dione, and 6,6′-dimethyl morpholine-2,5-dione, and said copolyester has one or more carboxyl group per chain to which is ionically or covalently bound a polyamino acid having at least five hydrogen-bearing amine group(s) per chain, wherein the device is an absorbable suture or hemostatic bandage coated with the nitrogenous α-caprolactone copolyester.

Another aspect of this invention deals with the synthesis of the carboxyl-bearing copolyesters that are made by copolymerizing the cyclic monomers in the presence of one or more hydroxy acid initiator(s) selected from the group represented by glycolic, malic, tartaric, and citric acids.

### DETAILED DESCRIPTION OF THE PREFERRED EMBODIMENTS

Polyesters comprising predominantly α-caprolactone-derived sequences generally refer to polymers with caprolactone-derived sequences of greater than 80 mole percent of the monomer compositions from which the polymeric compositions of this invention. These are derived, predominantly from ε-caprolactone, because of the low melting temperature (Tₘ) and exceptionally low glass transition temperature (T_{g}) of the resulting copolyesters, which make them well suited for incorporation into medical devices to infer certain desirable properties. These include coatings which improve frictional properties, and tie-down characteristics of braided sutures. In addition to ε-caprolactone, the copolyesters of this invention are based on no more than 20 mole percent of one or more comonomer selected from the following group of cyclic monomers: lactide, glycolide, morpholine-2,5-dione, 6,6′-dimethyl morpholine-2,5-dione, and p-dioxanone. In constructing the nitrogenous copolyester compositions that exhibit autocatalyzed hydrolytic degradation, this invention deals with two primary approaches. In the first approach, the cyclic comonomers are polymerized in the presence of a basic initiator, such as triethanolamine, and a ring-opening catalyst, such as stannous octanoate, to provide, for instance, a monocentric triaxial copolyester with a basic tertiary nitrogen atom at the center of the triaxial chain. In the second approach, the cyclic comonomers are polymerized in the presence of a ring-opening polymerization catalyst, such as stannous octanoate, and one or more hydroxy acid initiator selected from the following group to yield an acid-bearing chain with one or more carboxylic group per chain: glycolic, tartaric, malic, and citric acids. To introduce the nitrogenous component to the carboxy-bearing chain, the prior art teaches the use of a basic amino acid, such as lysine or arginine, to react or covalently bond to the carboxylic group(s) of the polymer copolyester chain. In contrast, the present invention deals with the use of basic polyamino acids, not known to have any particular pharmacological activity, instead.

For the triaxial nitrogenous copolyester, the prior art limits the molecular weight of the copolymer to less than 20 kDa, to maximize their effective use as lubricious coating of medical devices, including absorbable sutures. However, the present invention deals with copolyesters having a molecular weight in excess of 20 kDa to allow the use of these copolyesters in applications requiring the formation of a coherent film or matrix for fibrous constructs with superior mechanical integrity as compared to those treated with the low molecular weight, lubricious surface coating of the prior art. Of the many applications of the triaxial nitrogenous copolyesters of the present invention, are those dealing with their use to coat and size micro-threads, having a diameter of less than 100 micron, wherein the threads are made of twisted or braided multifilament yarn. In using such high molecular copolyesters at a level of at least 10 percent add-on on sized multifilament yarn constructs (that is to adjoin the constituent filaments of the yarn as a mild adhesive), such as sutures, the treated constructs display improved mechanical integrity, lower friction coefficient that is associated with superior surface lubricity, while maintaining high engineering compliance due to the low T_{g} of the sizing agent, surprisingly, in spite of its high molecular weight.

To construct the nitrogenous compositions from the acid-bearing ε-caprolactone copolyesters, the prior art teaches the use of a basic amino acid, such as lysine or arginine, to bind ionically or covalently to the carboxyl group(s) of the copolyester. However, the present invention deals with binding a basic polyamino acid to the carboxylic-bearing chain to maximize the incorporation of the basic moieties to achieve not only faster autocatalyzed hydrolytic chain degradation, but also to provide an absorbable substrate with sufficiently high density of a positive charge at physiologic pH. An absorbable coating of a device having a high density, positive charge allows the use of the nitrogenous copolyester composition for many new and different types of applications, depending on the molecular weight of the copolyester and type and molecular weight of the original nitrogen-bearing component of the nitrogenous compositions. The polyamino acids suited for binding to the carboxyl-bearing copolyester can be one of more of those selected from the following group, wherein the molecular weight of said polyamino acid varies from about 1 kDa to about 500 kDa: polylysine, polyarginine, lysine/arginine copolymers, and copolymers of at least 50 molar percent of lysine or arginine. Applications of these nitrogenous compositions include ones dealing with (1) low molecular weight, nitrogenous coatings on absorbable, braided sutures to improve their tie-down characteristics and render the surface hemostatic so as to improve wound healing; (2) low or high molecular weight, nitrogenous copolyester coatings on absorbable or non-absorbable monofilament or braided sutures used in vascular surgery to improve the tie-down property and render the surface hemostatic to minimize or eliminate blood leakage through suture needle holes; (3) low or high molecular weight, nitrogenous coatings/sizing agents for absorbable fibrous components of hemostatic vascular devices intended for treating vascular defects; (4) low molecular, nitrogenous coatings for absorbable or non-absorbable fibrous hemostatic bandages; (5) low or high molecular weight coatings for metallic hemostatic microfilaments; (6) low molecular weight, nitrogenous copolyesters as hemostatic sealants for treating vascular wall defects; (7) low or high molecular weight, nitrogenous hemostatic coatings for absorbable or partially absorbable synthetic vascular grafts and allied devices; and (8) low or high molecular weight, nitrogenous hemostatic or cell-adhering coatings on absorbable scaffolds for tissue engineering.

A specific aspect of this invention deals with nitrogenous ε-caprolactone copolyesters containing one or more bioactive agent selected from the group of said agent known to (1) have antimicrobial properties; (2) activate tissue ingrowth; (3) exhibit anti-inflammatory properties; and (4) act as a mild anesthetic agent or pain relief agent.

The following examples illustrate the claimed invention and are in no way intended to limit its scope.

### EXAMPLE 1

### Preparation of High Molecular Weight Copolyester of Monocentric, Triaxial, Nitrogenous 95/5 ε-Caprolactone Glycolide Copolyester (T-P1)

ε-Caprolactone (71.5 g, 0.627 mole), glycolide (3.8 g. 0.0328 mole), triethanolamine (0.5576 g, 3.74 mmole), and stannous octanoate (0.624 mL of 0.2 M toluene solution, 0.1247 mmole), were charged into a predried glass reactor equipped for mechanical stirring and providing a dry nitrogen environment. The polymerization mixture was heated at 40° C. under reduced pressure for about 15 minutes and then purged with dry nitrogen. The polymerization was achieved by heating the reactants to 150° C. for 5 hours. The resulting polymer was cooled, isolated, and characterized for identity by NMR (in CDCl₃) and IR, molecular dimensions by Gel Permeation Chromatography (GPC) in dichloromethane (DCM), and thermal properties by Differential Scanning Calorimetry (DSC). Key analytical data can be summarized as follows: Tₘ=53° C. ΔH_{f}=69 J/g M_{w}(GPC in DCM)=53 kDa

### EXAMPLE 2

### Coating/Sizing of Absorbable Suture braid Using T-P1 from Example 1

An absorbable suture braid size 9-0 made of 5/95 poly(1-lactide-co-glycolide) was coated by threading through an acetone solution of T-P1 (from Example 1) in a coating/sizing bath with an exit leading to a drying chamber. The dried, coated/sized suture was wound and dried further under reduced pressure until a constant weight is achieved to determine the percent add-on. Using different coating/sizing agents, concentrations resulted in coated/sized sutures having the following add-on: Concentration of T-P1 in

| | | | | |
|---|---|---|---|---|
| Acetone: | 9 | 15 | 18 | 20 |
| Percent Add-on Sutures: | 17 | 26 | 39 | 54 |

### EXAMPLE 3

### Coating/Sizing of Absorbable Twisted Multifilament Yarn Using T-P1 from Example 1

Following a procedure similar to that described in example 2, a twisted multifilament yarn, made of 12 single filaments having an average diameter of 11 microns, was coated/sized with a 15 percent solution of T-P1. The dried coated/sized yarn was shown to have a 21 percent add-on.

### EXAMPLE 4

### Preparation of Low Molecular Weight, Carboxyl-bearing 95/5 Caiprolactone Glycolide Copolymer (P-2) Using Glycolic Acid as Initiator

ε-Caprolactone (136.98 g, 1.2016 mole), glycolide (7.32 g. 0.0631 mole), glycolic acid (8.4 g, 0.11 mole), and stannous octanoate (0.632 mL of 0.2 M toluene solution, 1.2664×10⁻⁴ mole), were charged into a predried glass reactor equipped for mechanical stirring and providing a dry nitrogen environment. The polymerization mixture was heated at 40° C. under reduced pressure for about 15 minutes and then purged with dry nitrogen. The polymerization was achieved by heating the reactants to 150° C. for 4 hours. The resulting polymer was cooled, isolated, and characterized for identity by NMR (in CDCl₃) and IR, molecular dimensions by Gel Permeation Chromatography (GPC) in dichloromethane (DCM), and thermal properties by Differential Scanning Calorimetry (DSC). Key analytical data can be summarized as follows: Tₘ=48° C. ΔH_{f}=55 J/g M_{w}(GPC in DCM)=6.1 kDa

### EXAMPLE 5

### Preparation of Nitrogenous Copolyester Composition (N-P2) Using P-2 from Example 4

Copolymer P-2 (500 mg) from example 4 was dissolved in acetone (5 mL). To this solution was added an aqueous sodium bicarbonate solution (24.6 mg in 200 μL distilled water). A solution of poly-1-lysine hydrochloride (molecular weight of about 30 kDa) in distilled water (50 mg in 500 μL) was prepared and added while stirring to the solution of P-2 containing the sodium bicarbonate; after completing the addition, the reaction mixture was stirred at room temperature for 1 hour after which substantial precipitation of the polylysine/P-2 salt was noted. Excess distilled water was then added to complete the salt precipitation. The solid product was isolated after three cycles of centrifugation and rinsing with distilled water. The solid product was dried at room temperature under reduced pressure (0.1 mm Hg) until a constant weight is realized. The dried product was first analyzed by infrared (using a film cast from trifluoroethanol) and elemental analysis for nitrogen. The results suggest about 50 percent incorporation of the poly-1-lysine in P-2. Thermal properties of the product were determined by differential scanning calorimetry (DSC). The results indicated a Tₘ=52° C. and ΔH_{f}=81 J/g.

### EXAMPLE 6

### Coating/Sizing of Suture Braid Using N-P2 from Example 5

Following a procedure similar to that described in Example 2, a size 9-0 braided suture made of 5/95 poly(1-lactide-co-glycolide) was coated with a solution of N-P2 (from Example 5) in trifluoroethanol (TFE). Using different concentrations of the N-P2 solution in TFE resulted in the following percent add-on: Concentration of N-P2 in TFE,

| | | |
|---|---|---|
| percent: | 11.2 | 16.8 |
| Add-on, Weight percent: | 20 | 32 |

### EXAMPLE 7

### Preparation of High Molecular Weight Carboxyl-bearing 95/5 ε-Caprolactone/Glycolide Copolymer (P-3) Using Glycolic Acid as Initiator

Copolyester P-3 was prepared following an experimental scheme similar to that used in preparing, isolating, and characterizing copolymer P-2 (Example 4) with the exception of conducting the polymerization for 6.25 hours and using the following initial polymerization

| | |
|---|---|
| charge: | Caprolactone (71.3 g, 0.625 mole) |
| | Glycolide (3.8 g, 0.0328 mole) |
| | Glycolic acid (0.286 g, 3.76 mmole) |
| | Stannous Octanoate, (624 μL of 0.2 M toluene) |

Pertinent analytical data of P-3 are summarized as follows:

Tₘ=55° C., ΔH_{f}=77 J/g, M_{w}=73 kDa

### EXAMPLE 8

### Preparation of Nitrogenous Copolyester Composition (N-P3A) Using P-3 (from Example 7) and 1-Lysine

Copolymer P-3 (500 mg) from Example 7 was dissolved in acetone (5 mL). To this solution was added an aqueous solution of L-lysine (2.5 mg in 25μ distilled water). The mixed solution was dehydrated using anhydrous sodium sulfate (10 mg) and filtered. The resulting filtrate was used for coating/sizing braided suture as in Example 9.

### EXAMPLE 9

### Coating/Sizing of Suture Braid Using N-P3A (from Example 8)

The concentration of the acetone solution of N-P3A obtained in a scale-up run of Example 8 as filtrate was adjusted to obtain 3 percent (W/V). The resulting solution was used to coat/size a size 9-0 braided suture made of 5/95 poly(1-lactide-co-glycolide) following a similar procedure to that used in Example 3. The resulting coated/sized suture was shown to have an add-on of 18 percent. The 3 percent (W/V) solution was diluted further to attain a 2.7 percent (W/V) new solution. This was used to coat a second lot of the 9-0 suture, leading to a final add-on of 11 percent.

### EXAMPLE 10

### Preparation of Nitrogenous Copolyester Composition (N-P3B) using P-3 (from Example 7) and Poly-l-lysine Hydrochloride

Nitrogenous copolyester N-P3B was prepared and characterized following the same experimental schemes as those used for N-P2 in Example 5 with the exception of using the high molecular copolyester from Example 7. The nitrogenous product was shown to contain 5 percent of bound poly-1-lysine and exhibited a Tₘ of 56° C. and ΔH_{f} of 72 J/g.

### EXAMPLE 11

### Coating/Sizing of Suture Braid Using N-P3B (from Example 10)

Following a similar scheme to that described in Example 6, a size 9-0 braided suture made of 5/95 poly(1-lactide-co-glycolide) was coated/sized with a 10 percent (W/V) solution of N-P3B in TFE to achieve a dry add-on of 20 percent.

Many modifications and variations of this invention can be made without departing from its spirit and scope, as will be apparent to those skilled in the art. The specific embodiments described above are offered by way of example only, and the invention is to be limited only the terms of the appended claims, along with the full scope of equivalents to which such claims are entitled.

What is claimed is:

1. An absorbable, monocentric, triaxial, crystalline nitrogenous ε-caprolactone copolyester coating for a fibrous vascular device wherein the polymer molecular weight exceeds 20 kDa and the chain sequences consist of 85 to 97 percent caprolactone-derived units and 3 to 15 percent units derived from one or more of cyclic monomer selected from the group represented by glycolide, morpholine-2,5-dione, and 6,6′-dimethyl morpholine-2,5-dione, covalently linked to a central nitrogen of a tertiary amine.

2. An absorbable, monocentric, triaxial, crystalline nitrogenous ε-caprolactone copolyester coating as set forth in claim 1 wherein the fibrous vascular device comprises a micro-thread comprising absorbable, twisted multifilament yarn having at least 10 percent by weight of said coating.

3. An absorbable, monocentric, triaxial, crystalline nitrogenous ε-caprolactone copolyester coating as set forth in claim 1 wherein the fibrous vascular device comprises a micro-thread comprising absorbable, braided multifilament yarn having at least 10 percent by weight of said coating.

4. A medical device comprising an absorbable, crystalline nitrogenous ε-caprolactone copolyester wherein the polymer molecular weight is 2 to 40 kDa and chain sequences consisting of 85 to 97 percent of caprolactone-derived units and 3 to 15 percent units derived from one or more of cyclic monomer selected from the group represented by glycolide, morpholine-2,5-dione, and 6,6′-dimethyl morpholine-2,5-dione, and said copolyester has one or more carboxyl group per chain to which is ionically or covalently bound a polyamino acid having at least 5 hydrogen-bearing amine group(s) in sequence per chain.

5. A medical device comprising an absorbable, crystalline, nitrogenous ε-caprolactone copolyester as set forth in claim 4 wherein the polyamino acid is a polylysine or polyarginine having a molecular weight of at least 1 kDa and the said device is a hemostatic vascular sealant or capable of attaining hemostasis.

6. A medical device comprising an absorbable, crystalline, nitrogenous ε-caprolactone copolyester as set forth in claim 4 wherein the polyamino acid is a polyarginine having a molecular weight of at least 1 kDa.

7. A medical device comprising absorbable, crystalline, nitrogenous ε-caprolactone copolyester as set forth in claim 4 wherein said device is a hemostatic vascular sealant.

8. A medical device comprising an absorbable crystalline, nitrogenous, ε-caprolactone copolyester as set forth in claim 4 wherein said device is capable of attaining hemostasis.

9. A medical device comprising an absorbable, crystalline, nitrogenous ε-caprolactone copolyester as set forth in claim 4 wherein the said device is a hemostatic vascular device comprising a fibrous construct comprising a micro-thread comprising an absorbable, twisted multifilament yarn having at least 10 percent by weight of the nitrogenous copolyester as a coating.

10. A medical device comprising an absorbable, crystalline, nitrogenous ε-caprolactone copolyester as set forth in claim 4 wherein the said device is a hemostatic vascular device comprising a fibrous construct comprising a micro-thread comprising an absorbable, braided multifilament yarn having at least 10 percent by weight of the nitrogenous copolyester as a coating.

11. A medical device comprising an absorbable, crystalline, nitrogenous α-caprolactone copolyester as set forth in claim 4 wherein the device is an absorbable suture or hemostatic bandage coated with the nitrogenous α-caprolactone copolyester.

12. A medical device comprising an absorbable, crystalline, nitrogenous ε-caprolactone copolyester as set forth in claim 4 wherein the said device is an absorbable hemostatic bandage coated with the nitrogenous α-caprolactone polyester.

13. A medical device comprising an absorbable, crystalline, nitrogenous ε-caprolactone copolyester as set forth in claim 4 wherein the carboxyl-bearing copolyester is made by copolymerizing the cyclic monomers in the presence of one or more hydroxy acid initiator(s) selected from the group represented by glycolic, malic, tartaric, and citric acids.

14. A medical device comprising an absorbable, crystalline, nitrogenous ε-caprolactone copolyester as set forth in claim 5 wherein the carboxyl-bearing copolyester is made by copolymerizing the cyclic monomers in the presence of one or more hydroxy acid initiator(s) selected from the group represented by glycolic, malic, tartaric, and citric acids.

15. A medical device comprising absorbable, crystalline, nitrogenous ε-caprolactone copolyester as set forth in claim 5 wherein said device is a hemostatic vascular sealant.

16. A medical device comprising an absorbable crystalline, nitrogenous, ε-caprolactone copolyester as set forth in claim 5 wherein the said device is capable of attaining hemostasis.

17. A medical device comprising an absorbable, crystalline, nitrogenous E-caprolactone copolyester as set forth in claim 5 wherein the said device is a hemostatic vascular device comprising a fibrous construct comprising a micro-thread comprising an absorbable, twisted multifilament yarn having at least 10 percent by weight of the nitrogenous copolyester as a coating.

18. A medical device comprising an absorbable, crystalline, nitrogenous ε-caprolactone copolyester as set forth in claim 5 wherein the said device is a hemostatic vascular device comprising a fibrous construct comprising a micro-thread comprising an absorbable, braided multifilament yarn having at least 10 percent by weight of the nitrogenous copolyester as a coating.

19. A medical device comprising an absorbable, crystalline, nitrogenous ε-caprolactone copolyester as set forth in claim 5 wherein the device is an absorbable suture coated with the nitrogenous α-caprolactone copolyester.

20. A medical device comprising an absorbable, crystalline, nitrogenous ε-caprolactone copolyester as set forth in claim 5 wherein the said device is an absorbable hemostatic bandage coated with the nitrogenous ε-caprolactone polyester.
